(19) United States
(12) Patent Application Publication
(10) Pub. No.: US 2009/0071484 A1
(43) Pub. Date: Mar. 19, 2009

(54) ENDOTRACHEAL TUBE WITH INTRINSIC SUCTION & ENDOTRACHEAL SUCTION CONTROL VALVE

(76) Inventors: Paul William Black, Highlands, NC (US); Leila Meadows Martin, Highlands, NC (US)

Correspondence Address: GEORGE R. REARDON, 3356 STATION COURT, LAWRENCVILLE, GA, 30044, US

(21) Appl. No.: 11/855,995

(22) Filed: Sep. 14, 2007

(51) Int. Cl. A61M 16/04

(52) U.S. Cl. 128/207.14

(57) ABSTRACT

An improved endotracheal tube providing a built in suction channel for the removal of excessive secretions from the lumen of said tube and the tracheobronchial system is disclosed. Control valves for regulating the suction feature are also disclosed.

### BACKGROUND OF THE INVENTION

The technology described herein relates generally to endotracheal tubes used for intubating the trachea and like opening of human and animal bodies.

During general anesthesia the gasses introduced through the endotracheal tube and the tube itself create irritation in the tracheobronchial system resulting in secretions that must be suction removed periodically by the anesthesiologist to clear the airway.

In the current conventional system, this requires detaching the gas delivery apparatus from the end of the endotracheal tube so a smaller suction tube can be introduced and passed down the lumen of the endotracheal tube. When the suctioning is finished, the gas delivery apparatus must be reattached. Thus the flow of essential gasses to the patient is temporarily interrupted and some gasses undesirably escape into the operating room air breathed by the operating team, which can present a risk to the team personnel. Furthermore, the movement of the endotracheal tube and the insertion of yet another foreign object, the conventional suction tube itself, often stimulates involuntary patient muscle contractions and movements which can cause bleeding or otherwise interfere with surgical maneuvers.

### BRIEF SUMMARY OF THE INVENTION

The technology described herein improves upon existing endotracheal tubes by providing a built in suction channel whereby secretions that accumulate within the tube or beyond the distal end of the tube (the end positioned inside the patient) can be removed with minimal effort and minimal disruption of the gas delivery system.

In an exemplary embodiment an improved endotracheal tube is comprised of an outer tube the diameter of which approximates the inner diameter of the passage to be intubated. A separate suction channel runs from the proximal end of the outer tube (the end of the tube outside of the patient) to the distal end of the tube. An opening at the distal end of the suction channel and periodic openings along the length of the channel allow fluids to be suctioned into the channel from the area past the distal end of the outer tube and from the area within the lumen of the outer tube.

An extension tube in fluid communication with the suction channel extends outward from the proximal end of the outer tube. In an exemplary configuration the extension tube is connected to the inlet port of a valve means and the outlet port of the valve means is connected to an external suction apparatus commonly found in operating theatres. Both the endotracheal tube and the valves are intended to be single use devices.

Two embodiments of valve means for regulating the flow of fluids through the suction channel are disclosed. One valve opens when an operator depresses a push button and the other opens when an operator turns a knob. Both valve examples are designed to automatically close when the operator releases them. Both valve examples have a suction bypass feature whereby when the suction feature is not in use, room air is sucked through a vent hole in the valve body and into the suction system.

### BRIEF DESCRIPTION OF THE SEVERAL VIEWS OF THE DRAWING

The technology described herein will be better understood by reading the detailed description of the invention with reference to the accompanying drawing figures, in which like reference numerals denote similar structure and refer to like elements throughout, and in which:

FIG. 1 is a perspective view of the improved endotracheal tube.

FIG. 2 is a cross-sectional view of the endotracheal tube just below the cuff.

FIG. 3 is a side view of the endotracheal tube just below the cuff.

FIG. 4A is a front view and FIG. 4B and FIG. 4C are side views of the first valve means.

FIG. 5A is a front cross-sectional view and FIG. 5B is a side cross-sectional view of the first valve means in its closed position.

FIG. 5C is a cross-sectional view of the stem component of the first valve means.

FIG. 6A is a front cross-sectional view and FIG. 6B is a side cross-sectional view of the first valve means in its open position.

FIG. 7A is a front view of the second valve means in its closed position.

FIG. 7B is a front cross-sectional view, FIG. 7C is a mid side cross-sectional view, and FIG. 7D is an end side cross-sectional view of the second valve means in its closed position.

FIG. 8A is a front view of the second valve means in its open position.

FIG. 8B is a front cross-sectional view, FIG. 8C is a mid side cross-sectional view and FIG. 8D is an end side cross-sectional view of the second valve means in its open position.

### DETAILED DESCRIPTION OF THE INVENTION

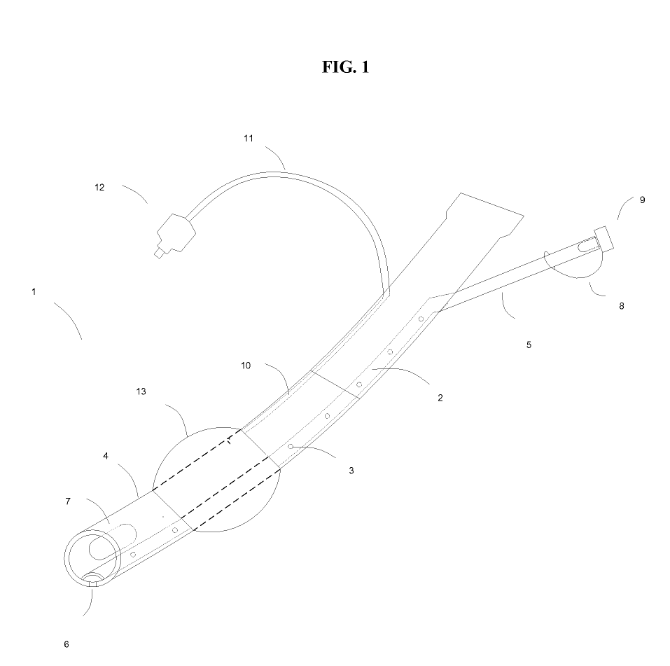

In describing the preferred and other embodiments of the technology described herein, as illustrated in FIGS. 1-8, specific terminology is employed for the sake of clarity. The invention, however, is not intended to be limited to the specific terminology so selected, and it is to be understood that each specific element includes all technical equivalents that operate in a similar manner to accomplish similar functions.

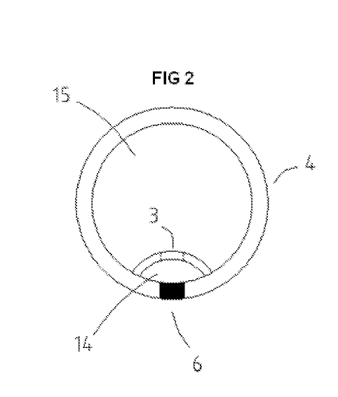

Referring now to FIG. 1 and FIG. 2, illustrated therein is an embodiment of an improved endotracheal tube with intrinsic suction 1. An outer tube 4 has an outer diameter that approximates the inner diameter of the passage to be intubated. A built in suction channel 2 runs through the outer tube. A plurality of a side hole 3 allows the movement of fluids from the outer tube lumen 15 into the suction channel lumen 14. A suction extension tube 5 is in fluid communication with the suction channel 2 and extends beyond the outer tube 4. An end cap 9 is used to close off the end of the suction extension tube 5 when the suction feature is not in use. A tether 8 attaches the end cap 9 to the extension tube 5.

An inflatable cuff 13 common to existing endotracheal tubes is positioned near the end of the tube. An air passageway 10, a filler valve 12 and inflation extension tube 11 allows air to be pumped into the inflatable cuff 13. A Murphy's Eye hole 7 is also common to existing endotracheal tubes and is positioned near the end of the outer tube 4. A radiopaque strip 6 runs the length of the outer tube 4 underneath the suction channel 2.

FIG. 2 illustrates a cross-sectional view of the endotracheal tube just below the cuff. A plurality of side hole 3 allows fluids to be sucked from the outer tube lumen 15 into the suction channel lumen 14 when the suction feature is activated.

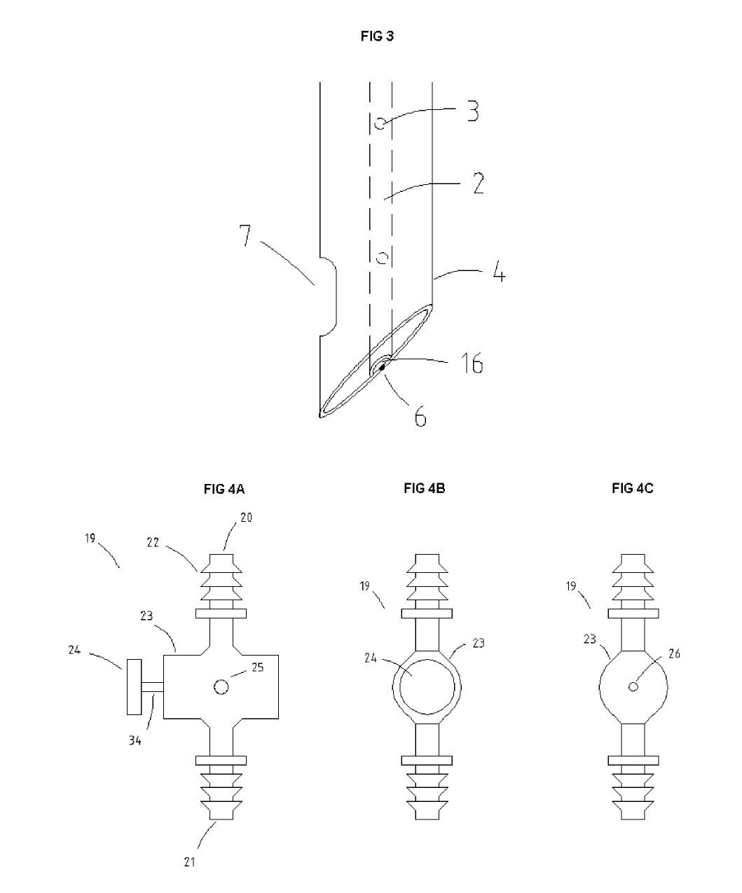

FIG. 3 illustrates a side view from the concave side of the end of the endotracheal tube just below the cuff. An end hole 16 at the end of the suction channel 2 allows fluids to be sucked into the suction channel 2 from the area beyond the end of the tube.

Referring now to FIG. 4A, illustrated therein is a front view of the first valve means 19 for the regulation of the flow of fluids through the suction channel 2. A fluid outlet port 20 and a fluid inlet port 21 extend outwards from the valve body 23. A hose connected to a suction system commonly found in operating environments will slide over the outlet port 20. The extension tube of the endotracheal tube's suction channel 2 will slide over the inlet port 21. A plurality of a ridge 22 on the outlet and inlet ports create an airtight seal between the hoses and the ports. A vent hole 25 in the front of the valve body allows air from the surrounding room to be sucked into the suction system when the valve is in the closed position. An additional vent hole (not shown in this drawing) is also on the back side of the valve body. A push button 24 controls the operation of the valve. A stem 34 connects the push button to a sliding element within the valve body 23.

FIG. 4B shows a side view of the first valve means 19 from the side facing the push button 24.

FIG. 4C shows another side view of the first valve means 19 from the side opposite the push button. A vent hole 26 allows air to move in and out of the valve body 23. This allows a reciprocatable element within the valve body to slide back and forth without creating a vacuum that could restrict the movement of the element.

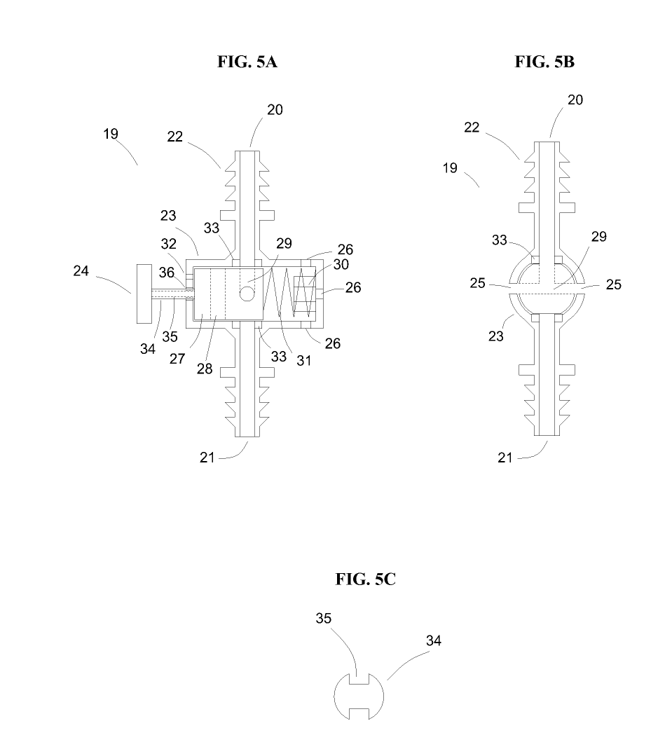

FIG. 5A illustrates a cross-sectional view of the first valve means 19 showing the reciprocatable element 27 in its closed position. Two fluid passageways are disposed within the reciprocatable element 27. A suction passageway 28 has one opening at the bottom of the reciprocatable element 27 and one opening at the top. A suction bypass passageway 29 has one opening at the top of the reciprocatable element 27 and two openings on the sides of the reciprocatable element 27. A spring 31 keeps the valve in the closed position until an operator depresses the push button 24. Silicone washers 33 provide airtight seals between the reciprocatable element 27 and both the inlet port 21 and the outlet port 20. A plurality of a vent hole 26 on the side of the valve body 23 opposite the push button and a vent hole 32 on the side of the body (end plate) under the push button allow air to move into the space between the reciprocatable element 27 and the valve body 23 preventing a vacuum for forming that could restrict the movement of the reciprocatable element 27. Grooves 35 in the stem 34 align with ridges 36 in the hole in the end plate through which the push button stem slides. The engagement of the grooves and ridges keep the stem 34 and reciprocatable element 27 from rotating out of alignment. A bumper block 30 prevents the reciprocatable element 27 from being pushed beyond the open position. The bumper block 30 has a hole in the middle of it to allow air to pass through it.

FIG. 5B illustrates a side mid-cross-sectional view of the valve means 19 in its closed position. The suction bypass passageway 29 is in fluid communication with the outlet port 20 and the two side vent holes 25 on each side of the valve body 23.

FIG. 5C illustrates a mid-cross-sectional view of the stem 34 connecting the push button with the reciprocatable element. Grooves in the stem 35 align with ridges attached to the valve body.

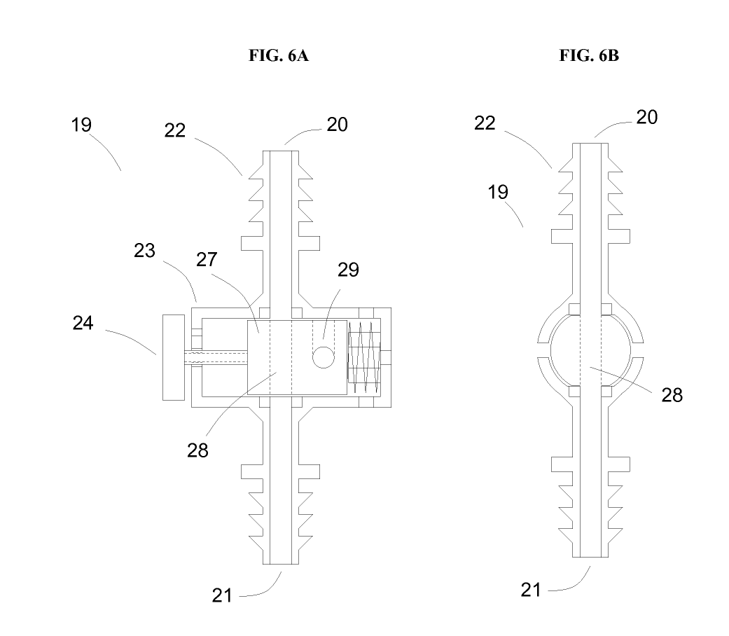

FIG. 6A illustrates a front cross-sectional view of the first valve means 19 with the push button depressed and the reciprocatable element 27 in its open position. In this position the suction passageway 28 is in fluid communication with the inlet port 21 and the outlet port 20 and the suction feature is activated.

FIG. 6B illustrates a side mid-cross-sectional view of the valve means 19 in its open position.

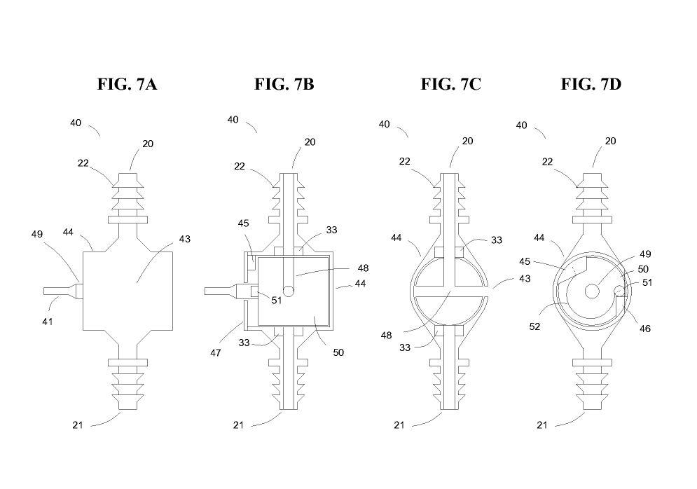

FIG. 7A illustrates a front view of a second valve means 40 in its closed position. This valve utilizes a rotatable knob 41 to turn a rotatable element disposed within the valve body 44. A knob stem 49 connects the rotatable knob 41 to the rotatable element. An inlet port 21 and an outlet port 20 extend outward from the valve body 44. A vent hole 43 represented in this drawing as a dotted line is positioned on the back of the valve body 44.

FIG. 7B illustrates a cross-sectional view of the valve means 40 in its closed position exposing a rotatable element 50. A fluid passageway 48 is disposed within the rotatable element 50. A spring anchor block 45 is attached to the valve body 44 and a spring attachment post 51 is attached to the rotatable element 50. A spring not shown in this figure connects the anchor block 45 and attachment post 51 and keeps the rotatable element 50 in its closed position when the suction feature is not needed. A stop block (not shown in this figure) attached to the valve body keeps the attachment post 51 and rotatable element 50 from moving beyond the valves closed position and within the ninety degree rotation arc. The spring anchor block 45 and the stop block also serve as spacers between the end of the rotatable element and the body end plate 47 which encloses the inner components of the valve 40.

FIG. 7C illustrates a side mid-cross-sectional view of the second valve means 40 in its closed position. This view illustrates that the fluid passageway 48 is comprised of two separate passageways joined together and has three openings. In this closed position one opening is in fluid communication with the outlet port 20 and another one is in fluid communication with the back vent hole 43. A silicone washer 33 forms an airtight seal between the rotatable element 50 and the outlet port 20.

FIG. 7D illustrates a side view of the second valve means 40 without the endplate and the knob. A spring 52 is attached to the spring anchor block 45 and to the spring attachment post 51. A stop block 46 keeps the attachment post 51 and the rotatable element 50 from moving beyond the valve's closed position.

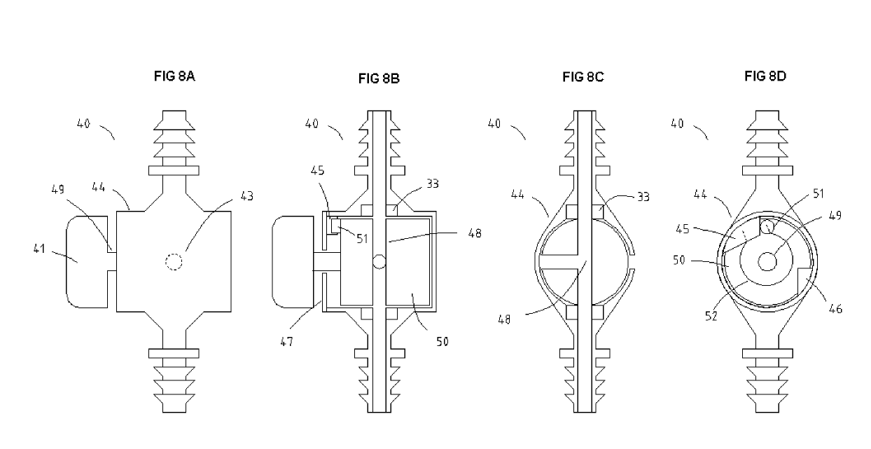

FIG. 8A illustrates a front view of the second valve means 40 shown in FIG. 7A where its knob 41 has been rotated ninety degrees from its closed position into its open position.

FIG. 8B illustrates a front cross-sectional view of the second valve means 40 shown in FIG. 7B where the rotatable element 50 and knob 41 have been rotated ninety degrees into in its open position. In this position one of the openings of the fluid passageway 48 is in fluid communication with the outlet port 20 and one of the openings is in fluid communication with the inlet port 21.

FIG. 8C illustrates a side mid-cross-sectional view of the second valve means 40 in its open position.

FIG. 8D illustrates a side view of the second valve means 40 without the endplate and the knob where an operator has rotated the rotatable element 50 ninety degrees into its open position. The spring anchor block 45 also functions as a stop block and keeps the rotatable element from moving past its open position. The spring 52 is compressed in this position and it will force the rotatable element 50 back into its closed position when the operator releases the knob.

Having thus described exemplary embodiments of the technology described herein, it should be noted by those skilled in the art that the within disclosures are exemplary only, and that various other alternatives, adaptations, and modifications may be made within the scope of the technology described herein. Accordingly, the technology described herein is not limited to the specific embodiments illustrated herein, but is limited only by the following claims.

What is claimed is:

1. An endotracheal tube with intrinsic suction, the endotracheal tube comprising:
an outer tube, the outer tube having a proximal end, a distal end, and a lumen, wherein the outer tube approximates an inner diameter of a body passage to be intubated; and
a suction channel integrally formed within the lumen of the outer tube, the suction channel spanning from the proximal end to the distal end of the outer tube and having an opening at the distal end and a plurality of openings along a length of the suction channel, the suction channel configured to allow a plurality of fluids to be suctioned into the suction channel from an area surrounding the distal end of the outer tube and from an area within the lumen of the outer tube.

2. The endotracheal tube of claim 1, further comprising:
an extension tube, fluidly coupled to the suction channel and outwardly extending from the proximal end of the outer tube, to provide a removal path out of the body passage for the plurality of fluids suctioned into the suction channel.

3. The endotracheal tube of claim 2, further comprising:
an end cap selectively placed on a proximal end of the extension tube to close the proximal end of the extension tube; and
a tether to connect the end cap to the extension tube.

4. The endotracheal tube of claim 2, further comprising:
a valve configured to regulate a flow of the plurality fluids suctioned into the suction channel and out of the body passage, the valve having a fluid inlet port and a fluid outlet port configured for attachment to the extension tube and an external suction device.

5. The endotracheal tube of claim 1, further comprising:
an inflatable cuff, positioned near the distal end of the outer tube;
an air passageway located within the lumen of the outer tube and fluidly coupled to the inflatable cuff,
an inflation extension tube fluidly coupled to the air passageway in the lumen of the outer tube and outwardly extending from the proximal end of the outer tube; and
a filler valve coupled to a proximal end of the inflation extension tube to regulate the flow of air entering or exiting the inflation extension tube and the inflatable cuff.

6. The endotracheal tube of claim 1, further comprising:
a radiopaque strip located within the outer tube in a portion of the outer tube located underneath the suction channel and extending longitudinally from the distal end of the outer tube to the proximate end of the outer tube to disallow the passage of radiation.

7. The endotracheal tube of claim 1, further comprising:
an opening in the outer tube located between a tip of the endotracheal tube and an inflatable cuff to assist in ventilation and help avoid complete endotracheal tube obstruction.

8. The endotracheal tube of claim 4, wherein the valve further comprises:
a valve body;
a push-button coupled to the valve body to regulate an operation of the valve;
the fluid outlet port and the fluid inlet port located on the valve body, the fluid outlet port fluidly coupled to an external suction device and the fluid inlet port fluidly coupled to an extension tube fluidly coupled to the suction channel;
a plurality of ridges located on the fluid outlet port and the fluid inlet port to create a seal when in use; and
one or more vent holes located on the valve body to allow air from a surrounding area to be drawn into the valve body when the valve is in a closed position and to provide a reciprocatable element the ability to operate within the valve without creating a vacuum.

9. The endotracheal tube of claim 8, wherein the reciprocatable element further comprises:
a first fluid passageway, disposed within the reciprocatable element, to provide a suction passageway; and
a second fluid passageway, disposed within the reciprocatable element, to provide a suction bypass passageway.

10. The endotracheal tube of claim 8, further comprising:
a stem disposed between the push button and a sliding element that moves within the valve body, coupling the push button to the sliding element;
a plurality of grooves located on an edge surface of the stem; and
a plurality of groves located in the valve body where the stem is received, to provide the stem and reciprocatable element from rotating out of alignment.

11. The endotracheal tube of claim 8, further comprising:
a spring to keep the valve closed until an operator depresses the push button to activate suction into the suction channel of the endotracheal tube.

12. The endotracheal tube of claim 8, further comprising:
a plurality of silicone washers to provide a seal between the reciprocatable element and the fluid outlet port and between the reciprocatable element and the fluid inlet port.

13. The endotracheal tube of claim 8, further comprising:
a plurality of vent holes located on a side of the valve body opposite the push button; and
a push button vent hole located on a side of the valve body with the push button, to allow air to move into a space between the reciprocatable element and the valve body and to prevent a formation of a vacuum that restricts a movement of the reciprocatable element.

14. The endotracheal tube of claim 8, further comprising:
a bumper block located within the valve body on a side of the valve body opposite the push button and the reciprocating element to prevent the reciprocating element from being pushed beyond an open position; and
a hole located in the bumper block to allow air to pass through the hole.

15. The endotracheal tube of claim 4, wherein the valve further comprises:
a valve body;
a rotatable turn-knob to turn a rotatable element disposed within the valve body, the rotatable turn-knob coupled to the rotatable element with a knob stem, to regulate an operation of the valve;
the fluid outlet port and the fluid inlet port located on and fluidly coupled to the valve body;
a plurality of ridges located on the fluid outlet port and the fluid inlet port to create a seal when in use; and
one or more vents holes located on the valve body to allow air from a surrounding area to be drawn into the valve body when the valve is in a closed position and to provide a rotatable element to operate within the valve without creating a vacuum.

16. The endotracheal tube of claim 15, further comprising:
a fluid passageway disposed with the rotatable element.

17. The endotracheal tube of claim 15, further comprising:
a spring anchor block disposed within the valve body;
a spring attachment post disposed within the valve body and attached to the rotatable element; and
a spring disposed within the valve body and coupled to the spring anchor block and the spring attachment post to keep the rotatable element in a closed position when a suction feature is not needed.

18. The endotracheal tube of claim 15, further comprising:
a stop block disposed within the valve body to prevent the attachment post and the rotatable element from moving beyond a closed position for the valve.

19. The endotracheal tube of claim 15, further comprising:
a plurality of silicone washers to provide a seal between the rotatable element and the fluid outlet port.

20. A method of placing an endotracheal tube with intrinsic suction within a patient, the method comprising:
providing a preassembled and packaged endotracheal tube with an integrally formed suction channel having an opening at a distal end and a plurality of openings along a length of the suction channel, the suction channel configured to allow a plurality of fluids to be suctioned into the suction channel;
placing the endotracheal tube within the patient;
ventilating the patient; and
utilizing the suction channel as necessary to remove accumulated fluid secretions.

21. The method of placing an endotracheal tube with intrinsic suction within a patient of claim 20, further comprising:
providing an extension tube, fluidly coupled to the suction channel and outwardly extending from a proximal end of the endotracheal tube, to provide a removal path out of the body passage for the plurality of fluids suctioned into the suction channel.

22. The method of placing an endotracheal tube with intrinsic suction within a patient of claim 20, further comprising:
providing an end cap to be selectively placed on a proximal end of the extension tube to close the proximal end of the extension tube; and
utilizing a tether to connect the end cap to the extension tube.

23. The method of placing an endotracheal tube with intrinsic suction within a patient of claim 20, further comprising:
utilizing a valve configured to regulate a flow of the plurality fluids suctioned into the suction channel and out of the body passage, the valve having a fluid inlet port and a fluid outlet port configured for attachment to the extension tube and an external suction device.

24. The method of placing an endotracheal tube with intrinsic suction within a patient of claim 23, further comprising:
providing a valve body; and
depressing a push-button coupled to the valve body to regulate an operation of the valve.

25. The method of placing an endotracheal tube with intrinsic suction within a patient of claim 23, further comprising:
providing a valve body; and
rotating a rotatable turn-knob to turn a rotatable element disposed within the valve body, the rotatable turn-knob coupled to the rotatable element with a knob stem, to regulate an operation of the valve.
